(19) United States
(12) Patent Application Publication
(10) Pub. No.: US 2023/0010501 A1
(43) Pub. Date: Jan. 12, 2023

(54) TREATMENT OF PARKINSON'S DISEASE BY IMMUNE MODULATION AND REGENERATIVE MEANS

(71) Applicant: Therapeutic Solutions International, Inc., Oceanside, CA (US)

(72) Inventors: Thomas E. Ichim, Oceanside, CA (US); Timothy G. Dixon, Oceanside, CA (US); James Veltmeyer, Oceanside, CA (US); Kalina O'Connor, Oceanside, CA (US)

(73) Assignee: Therapeutic Solutions International, Inc., Oceanside, CA (US)

(21) Appl. No.: 17/858,869

(22) Filed: Jul. 6, 2022

Related U.S. Application Data
(60) Provisional application No. 63/218,582, filed on Jul. 6, 2021.

(51) Int. Cl. A61K 35/15

(52) U.S. Cl. CPC A61K 35/15; A61K 2035/122

(57) ABSTRACT

Disclosed are means, methods and compositions of matter for treatment Parkinson's Disease through concurrent immune modulation and regenerative means. In one embodiment Parkinson's Disease is treated by augmentation of T regulatory cell numbers and/or activity while concurrently providing regenerative cells such as mesenchymal stem cells, and/or dopamine secreting cells. In one embodiment administration of immunoglobulins such as IVIG together with low dose interleukin-2 and/or low dose naltrexone is disclosed as a preparatory means prior to administration of therapeutic cells such as stem cells. Other therapeutic means utilized in an adjuvant manner are also provided for hormonal rebalancing, transcranial magnetic stimulation, and deep brain stimulation.

### CROSS-REFERENCE TO RELATED APPLICATIONS

This application claims the benefit of priority to U.S. Provisional Application No. 63/218,582, filed Jul. 6, 2021, the contents of which are incorporated herein by reference in their entirety.

### FIELD OF THE INVENTION

The invention relates to the field of neurodegenerative diseases, more particularly the invention relates to the field of dopaminergic neuron loss, more particularly the invention relates to means of preventing, stabilizing and/or reversing Parkinson's Disease.

### BACKGROUND OF THE INVENTION

Parkinson's Disease (PD) is believed to be the second most common and fastest growing neurodegenerative disorder worldwide. At present, it affects 2-3% of individuals over the age of 65, which is expected to double by the year 2040 [2, 3]. PD presents with motor symptoms such as tremors and bradykinesia, and non-motor symptoms, such as disordered sleep and cognitive dysfunction. As there is no cure for PD, symptoms inevitably progress and inflict devastating consequences on individuals and on their families [4-6]. In common with Alzheimer and other neurodegenerative diseases, PD is biologically characterized by protein misfolding and the rampant death of neurons [7-10]. Specifically, PD is characterized by the aggregation of α-synuclein protein and the death of dopaminergic neurons in the midbrain substantia nigra (SN), although PD affects other neurotransmitter systems as well [11-16]. Neuroinflammation has been associated with PD both in animal models [17-20], and in autopsy samples of patients [21, 22].

The medical treatment of Parkinson's disease is directed to stopping, slowing down, reducing the extent of or minimizing the neurodegenerative process in nigrostriatal neurons (neuroprotective therapy) and eliminating the biochemical imbalance (symptomatic therapy). The main directions of symptomatic therapy in Parkinson's disease are to increase dopamine synthesis, or stimulate dopamine receptors activity and dopamine release from the presynaptic space, and to inhibit dopamine reuptake by presynaptic receptors and dopamine catabolism.

The gold standard in the pharmacological treatment of Parkinson's disease is provided by DOPA-containing substances such as levodopa. Levodopa is commonly administered in combination with carbidopa, which increases the half-life of levodopa. However, the efficacy of these agents decreases over time because of continuing degeneration of neurons in the substantia nigra.

### SUMMARY

Preferred embodiments are directed to methods of preventing, and/or stabilizing progression of, and/or reversing Parkinson's Disease comprising induction of immunomodulatory and regenerative activity in a patient in need of therapy.

Preferred methods include embodiments wherein said immune modulatory therapy reduces inflammation in said patient with Parkinson's Disease.

Preferred methods include embodiments wherein said immune modulation is enhance of number and/or activity of T regulatory cells.

Preferred methods include embodiments wherein said inflammation is associated with a reduction of cells expressing the transcription factor FoxP3 as compared to an age matched control subject.

Preferred methods include embodiments wherein said inflammation is associated with a reduction of cells expressing the cytokine interleukin-10 as compared to an age matched control subject.

Preferred methods include embodiments wherein said inflammation is associated with a reduction of cells expressing the cytokine interleukin-4 as compared to an age matched control subject.

Preferred methods include embodiments wherein said inflammation is associated with a reduction of cells expressing the cytokine interleukin-13 as compared to an age matched control subject.

Preferred methods include embodiments wherein said inflammation is associated with a reduction of cells expressing the cytokine interleukin-20 as compared to an age matched control subject.

Preferred methods include embodiments wherein said inflammation is associated with a reduction of cells expressing the cytokine interleukin-35 as compared to an age matched control subject.

Preferred methods include embodiments wherein said inflammation is associated with a decrease in T regulatory cells as compared to an age matched control.

Preferred methods include embodiments wherein said inflammation is associated with a decrease in myeloid suppressor cells as compared to an age matched control.

Preferred methods include embodiments wherein said inflammation is associated with a decrease in TIM-1 expressing B cells as compared to an age matched control.

Preferred methods include embodiments wherein said inflammation is associated with a decrease in interleukin-10 expressing B cells as compared to an age matched control.

Preferred methods include embodiments wherein said inflammation is associated with a decrease in B regulatory cells as compared to an age matched control.

Preferred methods include embodiments wherein said inflammation is associated with an increase in cells expressing interferon gamma as compared to an age matched control.

Preferred methods include embodiments wherein said inflammation is associated with an increase in cells expressing TNF-alpha as compared to an age matched control.

Preferred methods include embodiments wherein said inflammation is associated with an increase in cells expressing interleukin-1 as compared to an age matched control.

Preferred methods include embodiments wherein said inflammation is associated with an increase in cells expressing interleukin-2 as compared to an age matched control.

Preferred methods include embodiments wherein said inflammation is associated with an increase in cells expressing interleukin-6 as compared to an age matched control.

Preferred methods include embodiments wherein said inflammation is associated with an increase in cells expressing interleukin-8 as compared to an age matched control.

Preferred methods include embodiments wherein said inflammation is associated with an increase in cells expressing interleukin-11 as compared to an age matched control.

Preferred methods include embodiments wherein said inflammation is associated with an increase in cells expressing interleukin-12 as compared to an age matched control.

Preferred methods include embodiments wherein said inflammation is associated with an increase in cells expressing interleukin-15 as compared to an age matched control.

Preferred methods include embodiments wherein said inflammation is associated with an increase in cells expressing interleukin-17 as compared to an age matched control.

Preferred methods include embodiments wherein said inflammation is associated with an increase in cells expressing interleukin-18 as compared to an age matched control.

Preferred methods include embodiments wherein said inflammation is associated with an increase in cells expressing interleukin-21 as compared to an age matched control.

Preferred methods include embodiments wherein said inflammation is associated with an increase in cells expressing interleukin-23 as compared to an age matched control.

Preferred methods include embodiments wherein said inflammation is associated with an increase in cells expressing interleukin-27 as compared to an age matched control.

Preferred methods include embodiments wherein said inflammation is associated with an increase in cells expressing interleukin-33 as compared to an age matched control.

Preferred methods include embodiments wherein said inflammation is associated with an increase in NK cells as compared to an age matched control.

Preferred methods include embodiments wherein said inflammation is associated with an increase in NKT cells as compared to an age matched control.

Preferred methods include embodiments wherein said inflammation is associated with an increase in Th1 cells as compared to an age matched control.

Preferred methods include embodiments wherein said Th1 cells produce more interferon gamma as compared to naïve T cells.

Preferred methods include embodiments wherein said Th1 cells produce more interleukin-2 as compared to naïve T cells.

Preferred methods include embodiments wherein said Th1 cells produce more interleukin-7 as compared to naïve T cells.

Preferred methods include embodiments wherein said Th1 cells produce more interleukin-18 as compared to naïve T cells.

Preferred methods include embodiments wherein said Th1 cells express more STAT4 as compared to naïve T cells.

Preferred methods include embodiments wherein said Th1 cells express more Helios as compared to naïve T cells.

Preferred methods include embodiments wherein said inflammation is associated with an increase in Th17 cells as compared to an age matched control.

Preferred methods include embodiments wherein said Th17 cells express a higher level of RoR gamma as compared to naïve T cells.

Preferred methods include embodiments wherein said Th17 cells express a higher level of interleukin-6 receptor as compared to naïve T cells.

Preferred methods include embodiments wherein said Th17 cells express a higher level of interleukin-17 as compared to naïve T cells.

Preferred methods include embodiments wherein said Th17 cells express a higher level of interleukin-17F as compared to naïve T cells.

Preferred methods include embodiments wherein said inflammation is associated with an increase in NK cells as compared to naïve T cells.

Preferred methods include embodiments wherein said NK cells express CD56.

Preferred methods include embodiments wherein said NK cells express CD16.

### BRIEF DESCRIPTION OF THE DRAWINGS

FIG. 1 is a bar graph showing the effects of treated dendritic cells+MPTP on MAC-1 expression levels.

FIG. 2 is a bar graph showing the effects of treated dendritic cells+MPTP on tyrosine hydroxylase percentages.

### DETAILED DESCRIPTION OF THE INVENTION

In one embodiment the invention aims to reduce abnormal immunological responses occurring in the brain of patient's with Parkinson's. In one embodiment

In one embodiment of the invention, immunomodulation and/or regeneration is utilized together with levodopa (L-DOPA), a precursor of dopamine, which is able to cross the blood-brain barrier, while dopamine itself cannot [23-27]. In the central nervous system, levodopa is metabolized to dopamine by aromatic L-amino acid decarboxylase (herein “decarboxylase”) and increases dopamine levels in the brain, being therefore indicated for symptomatic treatment of Parkinson's disease. However, levodopa is also converted to dopamine in the peripheral tissues, i.e. outside the brain. In order to prevent peripheral formation of dopamine, in one embodiment a peripheral decarboxylase inhibitor such as carbidopa or benserazide is coadministered with levodopa. In another embodiment, a catechol-O-methyl transferase (COMT) inhibitor such as tolcapone or entacapone is coadministered along with levodopa and carbidopa to prevent synthesis of dopamine in peripheral tissue.

In one embodiment of the invention, stem cells are provided together with quercetin [28].

Disclosed are means of inducing a tolerogenic state in patients with Parkinson's Disease. The invention teaches that administration of immature dendritic cells, of autologous and/or allogeneic origin, provides an environment conducive to stimulation of cells which inhibit inflammation and stimulate regeneration of injured and/or damaged dopaminergic cells. In one embodiment of the invention, patients are identified as having risk of Parkinson's Disease based on typical clinical parameters and/or cytokine alterations. Immune association with Parkinson's Disease and means of predicting are disclosed in the following papers that are incorporated by reference [29-41].

Means of using immune based markers for quantifying pathology of Parkinson's Disease is known in the art and incorporated by reference. For example, in one study, specific molecular signatures in patients with (PMC) and without (WMC) motor complications. mRNA levels of CD4+T lymphocytes transcription factor genes TBX21, STAT1, STAT3, STAT4, STAT6, RORC, GATA3, FOXP3, and NR4A2 were measured from 40 PD patients, divided into two groups according to motor complications. Also, 40 age- and sex-matched healthy controls were enrolled. WMC patients had higher levels of STAT1 and NR4A2 (p=0.004; p=0.003), whereas in PMC it was found that higher levels of STAT6 (p=0.04). Also, a ROC curve analysis confirmed STAT1 and NR4A2 as feasible biomarkers to discriminate WMC (AUC=0.76, 95% CI 0.59-0.92, p=0.005; AUC=0.75, 95% CI 0.58-0.90, p=0.007). Similarly, STAT6 detected PMC patients (AUC=0.69, 95% CI 0.52-0.86, p=0.037). These results provide evidence of different molecular signatures in CD 4+T cells of PD patients with and without MC, thus suggesting their potential as biomarkers of MC development [42].

The invention, in some embodiments, teaches the application of Immunological tolerance to the condition of Parkinson's Disease. It is known that a cardinal feature of the immune system, is allowing for recognition and elimination of pathological threats, while selectively ignoring antigens that belong to the body.

Traditionally, autoimmune conditions or conditions associated with cytokine storm, such as ARDS are treated with non-specific inhibitors of inflammation such as steroids, as well as immune suppressive agents such as cyclosporine, 5-azathrioprine, and methotrexate. These approaches globally suppress immune functions and have numerous undesirable side effects.

Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. The same holds true for cytokine storm conditions such as sepsis, where overproduction of agents such as TNF-alpha result in vascular leakage, coagulopathy, and death. The invention provides the utilization of tolerance-induction in ARDS alone, or in combination with existing techniques.

The utilization of antigen-nonspecific and/or antigen specific immature dendritic cells in treatment of Parkinson's Disease allows for induction of a inhibitory immune response, which results in suppression of brain inflammation. In one embodiment said tolerogenic dendritic cells are pulsed with antigens associated with Parkinson's Disease.

To provide prophylactic and/or therapeutic interventions, in the area of Parkinson's Disease, the invention teaches that it is important to delete/inactivate the T cell clone that are associated with stimulation of inflammation, as well as to block innate immune elements. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [43]; the “mirror image”, T regulatory (Treg) cells are actually selected to live by encounter with self-antigens, and Treg cells that do not bind self antigens are deleted [44, 45]. In one embodiment of the invention, immature dendritic cells are administered in order to induce a state of immune modulation, including T regulatory cell generation by the immature dendritic cells. Utilization of immature dendritic cells to stimulate T regulatory cell proliferation and/or activity has been previously demonstrated and is incorporated by reference [46-52].

Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

The invention teaches that utilization of tolerogenesis may be applied to Parkinson's Disease. Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [53], antigen presentation in the context of PD1-L [54], and HLA-G interacting with immune inhibitory receptors such as ILT4 [55]. Accordingly, in some embodiments of the invention, the utilization of tolerogenic regimens is provided which mimic pregnancy associated tolerance. In one embodiment, such embodiments include fusion of tolerance promoting molecules with Parkinson's Disease associated antigens such as synuclein peptides. In other embodiments synuclein antigens are co-administered with tolerogenic molecules such as ILT-4, or IL-10, or HLA-G.

In pregnancy, “tolerogenic antigen presentation” occurs only through the indirect pathway of antigen presentation [56]. Other pathways of selective tolerogenesis in pregnancy include the stimulation of Treg cells, which have been demonstrated essential for successful pregnancy [57]. In the context of cancer, depletion of tumor specific T cells, while sparing of T cells with specificities to other antigens has been demonstrated by the tumor itself or tumor associated cells [58-61]. Additionally, Treg cells have been demonstrated to actively suppress anti-tumor T cells, perhaps as a “back up” mechanism of tumor immune evasion [62-64]. At a clinical level the ability of tumors to inhibit peripheral T cell activity has been associated in numerous studies with poor prognosis [65-67]. Oral tolerance is the process by which ingested antigens induce generation of antigen-specific TGF-beta producing cells (called “Th3” by some) [68-70], as well as Treg cells [71, 72]. Ingestion of antigen, including the autoantigen collagen II [73], has been shown to induce inhibition of both T and B cell responses in a specific manner [74, 75]. It appears that induction of regulatory cells, as well as deletion/anergy of effector cells is associated with antigen presentation in a tolerogenic manner [76]. Remission of disease in animal models of RA [77], multiple sclerosis [78], and type I diabetes [79], has been reported by oral administration of autoantigens. Furthermore, clinical trials have shown signals of efficacy of oral tolerance in autoimmune diseases such as rheumatoid arthritis [80], autoimmune uveitis [81], and multiple sclerosis [82]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

In some embodiments of the invention the generation of immature dendritic cells is performed by either coculture in vitro, or administration in vivo of T regulatory cells [83].

In some embodiments of the invention, alpha 1 antitrypsin is administered in order to induce tolerogenic dendritic cells in order to treat Parkinson's Disease. The use of this compound for stimulation of immature DC has been previously described and is incorporated by reference [84].

In one embodiment immature dendritic cells are administered to treat Parkinson's Disease. Identification of these two conditions can be made based on techniques which are known in the art, and the methods described herein can be used to reduce, inhibit or alleviate at least one symptom of the disease.

In some embodiments of the invention, administration of immature dendritic cells to prevention and/or treat Parkinson's Disease is performed using other agents. Some agents include Inhaled nitric oxide (iNO),

In one embodiment the invention teaches reduction of Inflammatory cytokines, especially tumor necrosis factor alpha (TNF) and interleukin 1-beta (IL-1), by administration of immature dendritic cells. It is known that these inflammatory cytokines are major mediators that can elicit changes in cell phenotype, especially causing a variety of morphological and gene expression changes in endothelial cells. With respect to coagulation, one of the clot-promoting and one of the inhibitory pathways seem especially prone to modulation by these cytokines. In one embodiment, administration of immature dendritic cells is performed in order to reduce potential for coagulopathy.

In one embodiment of the invention, immature dendritic cells are utilized as biological regulator of inflammation. In some circumstances, the invention provides administration of IVIG alone or together with immature dendritic cells for treatment of Parkinson's Disease. In other embodiments, addition of regenerative cells such as mesenchymal stem cells is described. In yet other embodiments utilization of T regulatory cells is discussed as a means of enhancing the tolerogenic environment while regenerative cells are administered.

It is known that physiological conditions, inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g. asthma, psoriasis, arthritis, rheumatoid arthritis, multiple sclerosis, inflammatory bowel disease and the like), septic shock and multiple organ failure. Importantly, these diverse disease states share common inflammatory mediators, such as cytokines, chemokines, inflammatory cells and other mediators secreted by these cells. In the context of the current invention immature dendritic cells are utilized to inhibit pathological inflammation while allow various aspects of the immune response to remain intact.

Generally, inflammatory conditions, infection-associated conditions or immune-mediated inflammatory disorders that may be prevented or treated by administration of the immature dendritic cells. Examples of such inflammatory conditions include sepsis-associated conditions, inflammatory bowel diseases, autoimmune disorders, inflammatory disorders and infection-associated conditions. It is also thought that cancers, cardiovascular and metabolic conditions, neurologic and fibrotic conditions can be prevented or treated by administration of the TLR3 antibody antagonists of the invention. Inflammation may affect a tissue or be systemic. Exemplary affected tissues are the respiratory tract, lung, the gastrointestinal tract, small intestine, large intestine, colon, rectum, the cardiovascular system, cardiac tissue, blood vessels, joint, bone and synovial tissue, cartilage, epithelium, endothelium, hepatic or adipose tissue. It is to be noted that immature dendritic cells are generated with the concept of addressing major issues associated with Parkinson's Disease. In some embodiments of the invention

In one embodiment of the invention, regenerative cells and/or immune modulation is utilized together with Xigris (activated protein C (APC)) [85], which exerts its effects by activating endothelial cell-protecting mechanisms mediating protection against apoptosis, stimulation of barrier function through the angiopoietin/Tie-2 axis, and by reducing local clotting [86-88]. Without being bound to theory, the activity of Xigris appears to be associated with its ability to prevent the endothelial dysfunction [89] associated with suppression of proinflammatory chemokines [90], prevention of endothelial cell apoptosis [91], and increased endothelial fibrinolytic activity [92, 93]. Some of the protective activities of Xigris have been ascribed to its ability to suppress NF-kB activation in endothelial cells [94, 95].

Several clinical studies have supported the possibility that ascorbic acid (AA) mediates a beneficial effect on endothelial cells, especially in the context of chronic stress. Accordingly, in one embodiment of the invention immature dendritic cells are utilized together with AA. Heitzer et al. [96] examined acetylcholine-evoked endothelium-dependent vaso-responsiveness in 10 chronic smokers and 10 healthy volunteers. While responsiveness was suppressed in smokers, administration of intra-arterial ascorbate was capable of augmenting reactivity: an augmentation evident only in the smokers. Endothelial stress induced in 17 healthy volunteers by administration of L-methionine led to decreased responsiveness to hyperemic flow and increased homocysteine levels. Oral AA (1 g/day) restored endothelial responsiveness [97]. Restoration of endothelial responsiveness by AA has also been reported in patients with insulin-dependent [98] and independent diabetes [99], as well as chronic hypertension [100]. In these studies AA was administered intraarterially or intravenously, and the authors proposed the mechanism of action to be increased nitric oxide (NO) as a result of AA protecting it from degradation by reactive oxygen species (ROS).

A closer look at the literature suggests that there are several general mechanisms by which AA may exert endothelial protective properties. The importance of basal production of NO in endothelial function comes from its role as a vasodilator, and an inhibitor of platelet aggregation [101, 102]. High concentrations of NO are pathological in SIRS due to induction of vascular leakage [103]. However, lack of NO is also pathological because it causes loss of microvascular circulation and endothelial responsiveness [104, 105]. Although there are exceptions, the general concept is that inducible nitric oxide synthase (iNOS) and neuronal nitric oxide synthase (nNOS) are associated with sepsis-induced pathologies, whereas eNOS is associated with protective benefits [106]. It is important to note that, while iNOS expression occurs in almost all major cells of the body in the context of inflammation, eNOS is constitutively expressed by the endothelium. AA administration decreases iNOS in the context of inflammation [107, 108], but appears to increase eNOS [109]. Thus, AA appears to increase local NO concentrations through: a) prevention of ROS-mediated NO inactivation [110, 111]; b) increased activity of endothelial-specific nitric oxide synthase (eNOS) [112], possibly mediated by augmenting bioavailability of tetrahydrobiopterin [113-118], a co-factor of eNOS [119]; and c) induction of NO release from plasma-bound S-nitrosothiols [109].

In addition to deregulation of NO, numerous other endothelial changes occur during Parkinson's Disease, including endothelial cell apoptosis, upregulation of adhesion molecules, and the procoagulant state [120]. AA has been reported to be active in modulating each of these factors. Rossig et al. reported that in vitro administration of AA led to reduction of TNF-alpha induced endothelial cell apoptosis [109]. The effect was mediated in part through suppression of the mitochondria-initiated apoptotic pathway as evidenced by reduced caspase-9 activation and cytochrome c release. To extend their study into the clinical realm, the investigators prospectively randomized 34 patients with NYHA class III and IV heart failure to receive AA or placebo treatment. AA treatment (2.5 g administered intravenously and 3 days of 4 g per day oral AA) Resulted in reduction in circulating apoptotic endothelial cells in the treated but not placebo control group [121]. Various mechanisms for inhibition of endothelial cell apoptosis by AA have been proposed including upregulation of the anti-apoptotic protein bcl-2 [122] and the Rb protein, suppression of p53 [123], and increasing numbers of newly formed endothelial progenitor cells [124].

AA has been demonstrated to reduce endothelial cell expression of the adhesion molecule ICAM-1 in response to TNF-alpha in vitro in human umbilical vein endothelial (HUVEC) cells (HUVEC) [125]. By reducing adhesion molecule expression, AA suppresses systemic neutrophil extravasation during sepsis, especially in the lung [126]. Other endothelial effects of AA include suppression of tissue factor upregulation in response to inflammatory stimuli [127], and effect expected to prevent the hypercoaguable state. Furthermore, ascorbate supplementation has been directly implicated in suppressing endothelial permeability in the face of inflammatory stimuli [128-130], which would hypothetically reduce vascular leakage. Given the importance of NF-kappa B signaling in coordinating endothelial inflammatory changes [131-133], it is important to note that AA at pharmacologically attainable concentrations has been demonstrated to specifically inhibit this transcription factor on endothelial cells [134]. Mechanistically, several pathways of inhibition have been identified including reduction of i-kappa B phosphorylation and subsequent degradation [135], and suppression of activation of the upstream p38 MAPK pathway [136]. In vivo data in support of eventual use in humans has been reported showing that administration of 1 g per day AA in hypercholesterolemic pigs results in suppression of endothelial NF-kappa B activity, as well as increased eNOS, NO, and endothelial function [137]. In another porcine study, renal stenosis was combined with a high cholesterol diet to mimic renovascular disease. AA administered i.v. resulted in suppression of NF-kappa B activation in the endothelium, an effect associated with improved vascular function [138].

An important factor in reports of clinical studies of AA is the difference in effects seen when different routes of administration are employed. Supplementation with oral AA appears to have rather minor effects, perhaps due to the rate-limiting uptake of transporters found in the gut. Indeed, maximal absorption of AA appears to be achieved with a single 200 mg dose [139]. Higher doses produce gut discomfort and diarrhea because of effects of ascorbate accumulation in the intestinal lumen [140]. This is why some studies use parenteral administration. An example of the superior biological activity of parenteral versus oral was seen in a study administering AA to sedentary men. Parenteral but not oral administration was capable of augmenting endothelial responsiveness as assessed by a flow-mediated dilation assay [141].

In some embodiments of the invention immature dendritic cells are administered together with mesenchymal stem cells. “Mesenchymal stem cell” or “MSC” in some embodiments refers to cells that are (1) adherent to plastic, (2) express CD73, CD90, and CD105 antigens, while being CD14, CD34, CD45, and HLA-DR negative, are of autologous and/or allogeneic origin, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

### Example

Decrease in Substantia Nigra Inflammation by Tol-DC and Preservation of Dopaminergic Neurons

Tol-DC (StemVacs) was generated by culture of umbilical cord adherent monocytes in GM-CSF 10 ng/ml and IL-4 (5 ng/ml) for 7 days. Cells were treated with 5 ng/ml IL-10 to generate (Tol-DC), whereas conventional DC were cells grown under identical conditions with no IL-10.

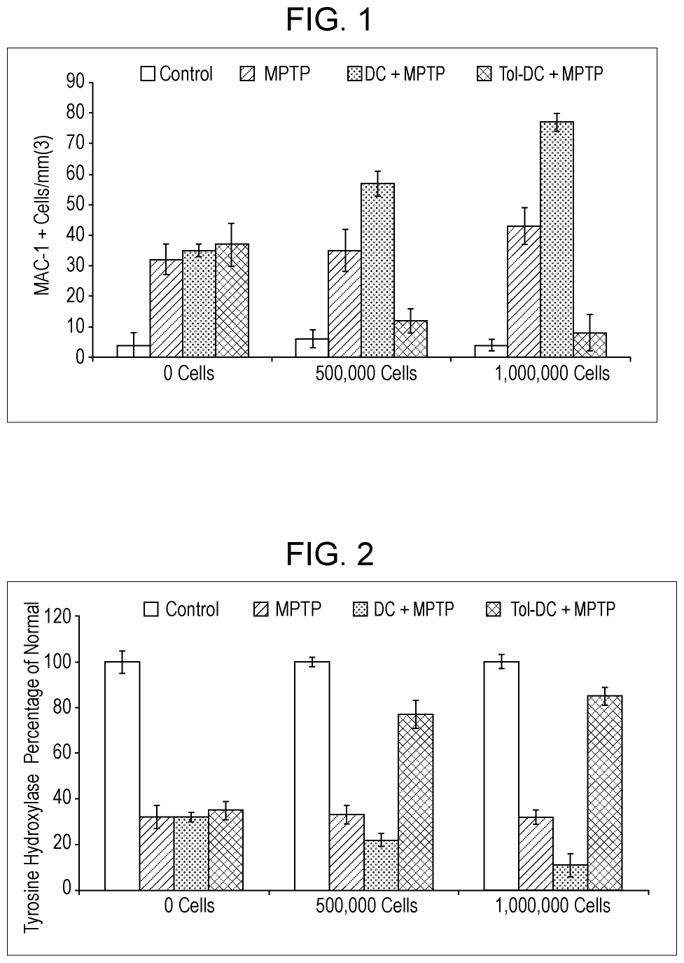

The cells were transferred i.v. at one and two weeks prior to intoxication with four 16 mg/kg doses of MPTP. Mice treated with PBS or MPTP alone served as controls. Two days after MPTP intoxication, mice were sacrificed, brains removed, frozen, and cryosectioned at 30 μm/section through the midbrain containing the substantia nigra. Sections were stained for Mac-1 expression by microglia. Tyrosine hydroxylase percentages were also measured. Results are shown in FIGS. 1 and 2.

### REFERENCES

1. Park, S. G., et al., Feline adipose tissue-derived mesenchymal stem cells pretreated with IFN-gamma enhance immunomodulatory effects through the PGE(2) pathway. J Vet Sci, 2021. 22(2): p. e16.
2. Poewe, W., et al., Parkinson disease. Nat Rev Dis Primers, 2017. 3: p. 17013.
3. Dorsey, E. R. and B. R. Bloem, The Parkinson Pandemic—A Call to Action. JAMA Neurol, 2018. 75(1): p. 9-10.
4. Armstrong, M. J. and M. S. Okun, Diagnosis and Treatment of Parkinson Disease: A Review. JAMA, 2020. 323(6): p. 548-560.
5. Reich, S. G. and J. M. Savitt, Parkinson's Disease. Med Clin North Am, 2019. 103(2): p. 337-350.
6. Baumann, C. R., et al., Body side and predominant motor features at the onset of Parkinson's disease are linked to motor and nonmotor progression. Mov Disord, 2014. 29(2): p. 207-13.
7. Kaur, R., S. Mehan, and S. Singh, Understanding multifactorial architecture of Parkinson's disease: pathophysiology to management. Neurol Sci, 2019. 40(1): p. 13-23.
8. Majd, S., J. H. Power, and H. J. Grantham, Neuronal response in Alzheimer's and Parkinson's disease: the effect of toxic proteins on intracellular pathways. BMC Neurosci, 2015. 16: p. 69.
9. Hinault, M. P., A. Farina-Henriquez-Cuendet, and P. Goloubinoff, Molecular chaperones and associated cellular clearance mechanisms against toxic protein conformers in Parkinson's disease. Neurodegener Dis, 2011. 8(6): p. 397-412.
10. Dong, Z., et al., Hsp70 gene transfer by adeno-associated virus inhibits MPTP-induced nigrostriatal degeneration in the mouse model of Parkinson disease. Mol Ther, 2005. 11(1): p. 80-8.
11. Teil, M., et al., Targeting alpha-synuclein for PD Therapeutics: A Pursuit on All Fronts. Biomolecules, 2020. 10(3).
12. O'Hara, D. M., S. K. Kalia, and L. V. Kalia, Methods for detecting toxic alpha-synuclein species as a biomarker for Parkinson's disease. Crit Rev Clin Lab Sci, 2020: p. 1-17.
13. Bernal-Conde, L. D., et al., Alpha-Synuclein Physiology and Pathology: A Perspective on Cellular Structures and Organelles. Front Neurosci, 2019. 13: p. 1399.
14. Koppen, J., et al., Amyloid-Beta Peptides Trigger Aggregation of Alpha-Synuclein In Vitro. Molecules, 2020. 25(3).
15. Faustini, G., et al., Alpha-Synuclein Preserves Mitochondrial Fusion and Function in Neuronal Cells. Oxid Med Cell Longev, 2019. 2019: p. 4246350.
16. Shams, R., N. L. Banik, and A. Hague, Calpain in the cleavage of alpha-synuclein and the pathogenesis of Parkinson's disease. Prog Mol Biol Transl Sci, 2019. 167: p. 107-124.
17. Miller, R. M., et al., Wild-type and mutant alpha-synuclein induce a multi-component gene expression profile consistent with shared pathophysiology in different transgenic mouse models of PD. Exp Neurol, 2007. 204(1): p. 421-32.
18. Smeyne, R. J., et al., Assessment of the Effects of MPTP and Paraquat on Dopaminergic Neurons and Microglia in the Substantia Nigra Pars Compacta of C57BL/6 Mice. PLoS One, 2016. 11(10): p. e0164094.
19. Sherer, T. B., et al., Mechanism of toxicity in rotenone models of Parkinson's disease. J Neurosci, 2003. 23(34): p. 10756-64.
20. Harms, A. S., et al., Delayed dominant-negative TNF gene therapy halts progressive loss of nigral dopaminergic neurons in a rat model of Parkinson's disease. Mol Ther, 2011. 19(1): p. 46-52.
21. Toulorge, D., A. H. Schapira, and R. Hajj, Molecular changes in the postmortem parkinsonian brain. J Neurochem, 2016. 139 Suppl 1: p. 27-58.
22. Moehle, M. S. and A. B. West, M1 and M2 immune activation in Parkinson's Disease: Foe and ally? Neuroscience, 2015. 302: p. 59-73.
23. Haddad, F., et al., Dopamine and Levodopa Prodrugs for the Treatment of Parkinson's Disease. Molecules, 2017. 23(1).
24. Helms, H. C. C., et al., Drug Delivery Strategies to Overcome the Blood-Brain Barrier (BBB). Handb Exp Pharmacol, 2020.
25. Lerner, R. P., et al., Levodopa-induced abnormal involuntary movements correlate with altered permeability of the blood-brain-barrier in the basal ganglia. Sci Rep, 2017. 7(1): p. 16005.
26. Friis, M. L., et al., Blood-brain barrier permeability of L-dopa in man. Eur J Clin Invest, 1981. 11(3): p. 231-4.
27. Alexander, G. M., et al., Effect of plasma levels of large neutral amino acids and degree of parkinsonism on the blood-to-brain transport of levodopa in naive and MPTP parkinsonian monkeys. Neurology, 1994. 44(8): p. 1491-9.
28. Boyina, H. K., et al., In Silico and In Vivo Studies on Quercetin as Potential Anti- Parkinson Agent. Adv Exp Med Biol, 2020. 1195: p. 1-11.
29. Dressman, D. and W. Elyaman, T Cells: A Growing Universe of Roles in Neurodegenerative Diseases. Neuroscientist, 2021: p. 10738584211024907.
30. Dong, L., et al., High Inflammatory Tendency Induced by Malignant Stimulation Through Imbalance of CD28 and CTLA-4/PD-1 Contributes to Dopamine Neuron Injury. J Inflamm Res, 2021. 14: p. 2471-2482.
31. MacMahon Copas, A. N., et al., The Pathogenesis of Parkinson's Disease: A Complex Interplay Between Astrocytes, Microglia, and T Lymphocytes? Front Neurol, 2021. 12: p. 666737.
32. Oliveira-Giacomelli, A., et al., Role of P2X7 Receptors in Immune Responses During Neurodegeneration. Front Cell Neurosci, 2021. 15: p. 662935.
33. Olson, K. E., et al., Safety, tolerability, and immune-biomarker profiling for year-long sargramostim treatment of Parkinson's disease. EBioMedicine, 2021. 67: p. 103380.
34. Chen, X., et al., Evidence for Peripheral Immune Activation in Parkinson's Disease. Front Aging Neurosci, 2021. 13: p. 617370.
35. Thome, A. D., et al., Ex vivo expansion of dysfunctional regulatory T lymphocytes restores suppressive function in Parkinson's disease. NPJ Parkinsons Dis, 2021. 7(1): p. 41.
36. Wang, B. Y., et al., Stress increases MHC-I expression in dopaminergic neurons and induces autoimmune activation in Parkinson's disease. Neural Regen Res, 2021. 16(12): p. 2521-2527.
37. Li, W., et al., Imbalance between T helper 1 and regulatory T cells plays a detrimental role in experimental Parkinson's disease in mice. J Int Med Res, 2021. 49(4): p. 300060521998471.
38. Olson, K. E., et al., Granulocyte-macrophage colony-stimulating factor mRNA and Neuroprotective Immunity in Parkinson's disease. Biomaterials, 2021. 272: p. 120786.
39. Williams, G. P., et al., CD4 T cells mediate brain inflammation and neurodegeneration in a mouse model of Parkinson disease. Brain, 2021.
40. George, S., et al., T Cells Limit Accumulation of Aggregate Pathology Following Intrastriatal Injection of alpha-Synuclein Fibrils. J Parkinsons Dis, 2021. 11(2): p. 585-603.
41. Harms, A. S., S. A. Ferreira, and M. Romero-Ramos, Periphery and brain, innate and adaptive immunity in Parkinson's disease. Acta Neuropathol, 2021. 141(4): p. 527-545.
42. Contaldi, E., et al., Expression of Transcription Factors in CD4+ T Cells as Potential Biomarkers of Motor Complications in Parkinson's Disease. J Parkinsons Dis, 2021. 11(2): p. 507-514.
43. Ramsdell, F. and B. J. Fowlkes, Clonal deletion versus clonal anergy: the role of the thymus in inducing self tolerance. Science, 1990. 248(4961): p. 1342-8.
44. Apostolou, I., et al., Origin of regulatory T cells with known specificity for antigen. Nat Immunol, 2002. 3(8): p. 756-63.
45. Aschenbrenner, K., et al., Selection of Foxp3+ regulatory T cells specific for self antigen expressed and presented by Aire+ medullary thymic epithelial cells. Nat Immunol, 2007. 8(4): p. 351-8.
46. Cong, Y., et al., Generation of antigen-specific, Foxp3-expressing CD4+ regulatory T cells by inhibition of APC proteosome function. J Immunol, 2005. 174(5): p. 2787-95.
47. Buckland, M., et al., Aspirin-treated human DCs up-regulate ILT-3 and induce hyporesponsiveness and regulatory activity in responder T cells. Am J Transplant, 2006. 6(9): p. 2046-59.
48. Jin, Y., et al., Induction of auto-reactive regulatory T cells by stimulation with immature autologous dendritic cells. Immunol Invest, 2007. 36(2): p. 213-32.
49. Gandhi, R., D. E. Anderson, and H. L. Weiner, Cutting Edge: Immature human dendritic cells express latency-associated peptide and inhibit T cell activation in a TGF-beta- dependent manner. J Immunol, 2007. 178(7): p. 4017-21.
50. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.
51. Zhang, X., et al., Generation of therapeutic dendritic cells and regulatory T cells for preventing allogeneic cardiac graft rejection. Clin Immunol, 2008. 127(3): p. 313-21.
52. Marguti, I., et al., Expansion of CD4+ CD25+ Foxp3+ T cells by bone marrow-derived dendritic cells. Immunology, 2009. 127(1): p. 50-61.
53. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.
54. D'Addio, F., et al., The link between the PDL1 costimulatory pathway and Th17 in fetomaternal tolerance. J Immunol, 2011. 187(9): p. 4530-41.
55. Kuroki, K. and K. Maenaka, Immune modulation of HLA-G dimer in maternal-fetal interface. Eur J Immunol, 2007. 37(7): p. 1727-9.
56. Erlebacher, A., et al., Constraints in antigen presentation severely restrict T cell recognition of the allogeneic fetus. J Clin Invest, 2007. 117(5): p. 1399-411.
57. Chen, T., et al., Self-specific memory regulatory T cells protect embryos at implantation in mice. J Immunol, 2013. 191(5): p. 2273-81.
58. Harimoto, H., et al., Inactivation of tumor-specific CD8(+) CTLs by tumor-infiltrating tolerogenic dendritic cells. Immunol Cell Biol, 2013. 91(9): p. 545-55.
59. Ney, J. T., et al., Autochthonous liver tumors induce systemic T cell tolerance associated with T cell receptor down-modulation. Hepatology, 2009. 49(2): p. 471-81.
60. Cheung, A. F., et al., Regulated expression of a tumor-associated antigen reveals multiple levels of T-cell tolerance in a mouse model of lung cancer. Cancer Res, 2008. 68(22): p. 9459-68.
61. Bal, A., et al., Rapid tolerization of virus-activated tumor-specific CD8+ T cells in prostate tumors of TRAMP mice. Proc Natl Acad Sci USA, 2008. 105(35): p. 13003-8.
62. Jacobs, J. F., et al., Regulatory T cells in melanoma: the final hurdle towards effective immunotherapy? Lancet Oncol, 2012. 13(1): p. e32-42.
63. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.
64. Donkor, M. K., et al., T cell surveillance of oncogene-induced prostate cancer is impeded by T cell-derived TGF-beta1 cytokine. Immunity, 2011. 35(1): p. 123-34.
65. Whiteside, T. L., Down-regulation of zeta-chain expression in T cells: a biomarker of prognosis in cancer? Cancer Immunol Immunother, 2004. 53(10): p. 865-78.
66. Whiteside, T. L., Signaling defects in T lymphocytes of patients with malignancy. Cancer Immunol Immunother, 1999. 48(7): p. 346-52.
67. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
68. Faria, A. M. and H. L. Weiner, Oral tolerance. Immunol Rev, 2005. 206: p. 232-59.
69. Weiner, H. L., Induction and mechanism of action of transforming growth factor-beta- secreting Th3 regulatory cells. Immunol Rev, 2001. 182: p. 207-14.
70. Fukaura, H., et al., Induction of circulating myelin basic protein and proteolipid protein- specific transforming growth factor-beta1-secreting Th3 T cells by oral administration of myelin in multiple sclerosis patients. J Clin Invest, 1996. 98(1): p. 70-7.
71. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.
72. Yamashita, H., et al., Overcoming food allergy through acquired tolerance conferred by transfer of Tregs in a murine model. Allergy, 2012. 67(2): p. 201-9.
73. Park, K. S., et al., Type II collagen oral tolerance; mechanism and role in collagen- induced arthritis and rheumatoid arthritis. Mod Rheumatol, 2009.
74. Womer, K. L., et al., A pilot study on the immunological effects of oral administration of donor major histocompatibility complex class II peptides in renal transplant recipients. Clin Transplant, 2008. 22(6): p. 754-9.
75. Faria, A. M. and H. L. Weiner, Oral tolerance: therapeutic implications for autoimmune diseases. Clin Dev Immunol, 2006. 13(2-4): p. 143-57.
76. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.
77. Thompson, H. S., et al., Suppression of collagen induced arthritis by oral administration of type II collagen: changes in immune and arthritic responses mediated by active peripheral suppression. Autoimmunity, 1993. 16(3): p. 189-99.
78. Song, F., et al., The thymus plays a role in oral tolerance induction in experimental autoimmune encephalomyelitis. Ann N Y Acad Sci, 2004. 1029: p. 402-4.
79. Hanninen, A. and L. C. Harrison, Mucosal tolerance to prevent type 1 diabetes: can the outcome be improved in humans? Rev Diabet Stud, 2004. 1(3): p. 113-21.
80. Wei, W., et al., A multicenter, double-blind, randomized, controlled phase III clinical trial of chicken type II collagen in rheumatoid arthritis. Arthritis Res Ther, 2009. 11(6): p. R180.
81. Thurau, S. R., et al., Molecular mimicry as a therapeutic approach for an autoimmune disease: oral treatment of uveitis-patients with an MHC peptide crossreactive with autoantigen—first results. Immunol Lett, 1997. 57(1-3): p. 193-201.
82. Weiner, H. L., et al., Double-blind pilot trial of oral tolerization with myelin antigens in multiple sclerosis. Science, 1993. 259(5099): p. 1321-4.
83. Onishi, Y., et al., Foxp3+ natural regulatory T cells preferentially form aggregates on dendritic cells in vitro and actively inhibit their maturation. Proc Natl Acad Sci USA, 2008. 105(29): p. 10113-8.
84. Ozeri, E., et al., alpha-1 antitrypsin promotes semimature, IL-10-producing and readily migrating tolerogenic dendritic cells. J Immunol, 2012. 189(1): p. 146-53.
85. Ely, E. W., G. R. Bernard, and J. L. Vincent, Activated protein C for severe sepsis. N Engl J Med, 2002. 347(13): p. 1035-6.
86. Dhainaut, J. F., et al., Drotrecogin alfa (activated) (recombinant human activated protein C) reduces host coagulopathy response in patients with severe sepsis. Thromb Haemost, 2003. 90(4): p. 642-53.
87. Minhas, N., et al., Activated protein C utilizes the angiopoietin/Tie2 axis to promote endothelial barrier function. FASEB J. 24(3): p. 873-81.
88. Loubele, S. T., H. M. Spronk, and H. Ten Cate, Activated protein C: a promising drug with multiple effects? Mini Rev Med Chem, 2009. 9(5): p. 620-6.
89. Grinnell, B. W. and D. Joyce, Recombinant human activated protein C: a system modulator of vascular function for treatment of severe sepsis. Crit Care Med, 2001. 29(7 Suppl): p. S53-60; discussion S60-1.
90. Brueckmann, M., et al., Activated protein C inhibits the release of macrophage inflammatory protein-1-alpha from THP-1 cells and from human monocytes. Cytokine, 2004. 26(3): p. 106-13.
91. Cheng, T., et al., Activated protein C blocks p53-mediated apoptosis in ischemic human brain endothelium and is neuroprotective. Nat Med, 2003. 9(3): p. 338-42.
92. van Hinsbergh, V. W., et al., Activated protein C decreases plasminogen activator- inhibitor activity in endothelial cell-conditioned medium. Blood, 1985. 65(2): p. 444-51.
93. Sakata, Y., et al., Activated protein C stimulates the fibrinolytic activity of cultured endothelial cells and decreases antiactivator activity. Proc Natl Acad Sci USA, 1985. 82(4): p. 1121-5.
94. Joyce, D. E. and B. W. Grinnell, Recombinant human activated protein C attenuates the inflammatory response in endothelium and monocytes by modulating nuclear factor- kappaB. Crit Care Med, 2002. 30(5 Suppl): p. S288-93.
95. Brueckmann, M., et al., Drotrecogin alfa (activated) inhibits NF-kappa B activation and MIP-1-alpha release from isolated mononuclear cells of patients with severe sepsis. Inflamm Res, 2004. 53(10): p. 528-33.
96. Heitzer, T., H. Just, and T. Munzel, Antioxidant vitamin C improves endothelial dysfunction in chronic smokers. Circulation, 1996. 94(1): p. 6-9.
97. Chambers, J. C., et al., Demonstration of rapid onset vascular endothelial dysfunction after hyperhomocysteinemia: an effect reversible with vitamin C therapy. Circulation, 1999. 99(9): p. 1156-60.
98. Timimi, F. K., et al., Vitamin C improves endothelium-dependent vasodilation in patients with insulin-dependent diabetes mellitus. J Am Coll Cardiol, 1998. 31(3): p. 552-7.
99. Ting, H. H., et al., Vitamin C improves endothelium-dependent vasodilation in patients with non-insulin-dependent diabetes mellitus. J Clin Invest, 1996. 97(1): p. 22-8.
100. Solzbach, U., et al., Vitamin C improves endothelial dysfunction of epicardial coronary arteries in hypertensive patients. Circulation, 1997. 96(5): p. 1513-9.
101. Gao, Y., The multiple actions of NO. Pflugers Arch. 459(6): p. 829-39.
102. Jackson, W. F., The endothelium-derived relaxing factor. J Reconstr Microsurg, 1989. 5(3): p. 263-71.
103. De Cruz, S. J., N. J. Kenyon, and C. E. Sandrock, Bench-to-bedside review: the role of nitric oxide in sepsis. Expert Rev Respir Med, 2009. 3(5): p. 511-21.
104. Tyml, K., F. Li, and J. X. Wilson, Septic impairment of capillary blood flow requires nicotinamide adenine dinucleotide phosphate oxidase but not nitric oxide synthase and is rapidly reversed by ascorbate through an endothelial nitric oxide synthase-dependent mechanism. Crit Care Med, 2008. 36(8): p. 2355-62.
105. Naseem, K. M., The role of nitric oxide in cardiovascular diseases. Mol Aspects Med, 2005. 26(1-2): p. 33-65.
106. Parratt, J. R., Nitric oxide in sepsis and endotoxaemia. J Antimicrob Chemother, 1998. 41 Suppl A: p. 31-9.
107. Wu, F., K. Tyml, and J. X. Wilson, Ascorbate inhibits iNOS expression in endotoxin- and IFN gamma-stimulated rat skeletal muscle endothelial cells. FEBS Lett, 2002. 520(1-3): p. 122-6.
108. Wu, F., J. X. Wilson, and K. Tyml, Ascorbate inhibits iNOS expression and preserves vasoconstrictor responsiveness in skeletal muscle of septic mice. Am J Physiol Regul Integr Comp Physiol, 2003. 285(1): p. R50-6.
109. Ulker, S., P. P. McKeown, and U. Bayraktutan, Vitamins reverse endothelial dysfunction through regulation of eNOS and NAD(P)H oxidase activities. Hypertension, 2003. 41(3): p. 534-9.
110. Peluffo, G., et al., Superoxide-mediated inactivation of nitric oxide and peroxynitrite formation by tobacco smoke in vascular endothelium: studies in cultured cells and smokers. Am J Physiol Heart Circ Physiol, 2009. 296(6): p. H1781-92.
111. May, J. M., Z. C. Qu, and X. Li, Ascorbic acid blunts oxidant stress due to menadione in endothelial cells. Arch Biochem Biophys, 2003. 411(1): p. 136-44.
112. Heller, R., et al., L-Ascorbic acid potentiates nitric oxide synthesis in endothelial cells. J Biol Chem, 1999. 274(12): p. 8254-60.
113. Nakai, K., et al., Ascorbate enhances iNOS activity by increasing tetrahydrobiopterin in RAW 264.7 cells. Free Radic Biol Med, 2003. 35(8): p. 929-37.
114. d'Uscio, L. V., et al., Long-term vitamin C treatment increases vascular tetrahydrobiopterin levels and nitric oxide synthase activity. Circ Res, 2003. 92(1): p. 88-95.
115. Toth, M., Z. Kukor, and S. Valent, Chemical stabilization of tetrahydrobiopterin by L- ascorbic acid: contribution to placental endothelial nitric oxide synthase activity. Mol Hum Reprod, 2002. 8(3): p. 271-80.
116. Patel, K. B., et al., Oxidation of tetrahydrobiopterin by biological radicals and scavenging of the trihydrobiopterin radical by ascorbate. Free Radic Biol Med, 2002. 32(3): p. 203-11.
117. Stone, K. J. and B. H. Townsley, The effect of L-ascorbate on catecholamine biosynthesis. Biochem J, 1973. 131(3): p. 611-3.
118. Huang, A., et al., Ascorbic acid enhances endothelial nitric-oxide synthase activity by increasing intracellular tetrahydrobiopterin. J Biol Chem, 2000. 275(23): p. 17399-406.
119. Schmidt, T. S. and N. J. Alp, Mechanisms for the role of tetrahydrobiopterin in endothelial function and vascular disease. Clin Sci (Lond), 2007. 113(2): p. 47-63.
120. Keel, M. and O. Trentz, Pathophysiology of polytrauma. Injury, 2005. 36(6): p. 691-709.
121. Rossig, L., et al., Vitamin C inhibits endothelial cell apoptosis in congestive heart failure. Circulation, 2001. 104(18): p. 2182-7.
122. Haendeler, J., A. M. Zeiher, and S. Dimmeler, Vitamin C and E prevent lipopolysaccharide-induced apoptosis in human endothelial cells by modulation of Bcl-2 and Bax. Eur J Pharmacol, 1996. 317(2-3): p. 407-11.
123. Saeed, R. W., T. Peng, and C. N. Metz, Ascorbic acid blocks the growth inhibitory effect of tumor necrosis factor-alpha on endothelial cells. Exp Biol Med (Maywood), 2003. 228(7): p. 855-65.
124. Fiorito, C., et al., Antioxidants increase number of progenitor endothelial cells through multiple gene expression pathways. Free Radic Res, 2008. 42(8): p. 754-62.
125. Mo, S. J., et al., Modulation of TNF-alpha-induced ICAM-1 expression, NO and H2O2 production by alginate, allicin and ascorbic acid in human endothelial cells. Arch Pharm Res, 2003. 26(3): p. 244-51.
126. Martin, W. J., 2nd, Neutrophils kill pulmonary endothelial cells by a hydrogen-peroxide- dependent pathway. An in vitro model of neutrophil-mediated lung injury. Am Rev Respir Dis, 1984. 130(2): p. 209-13.
127. Chen, Y. H., et al., Anti-inflammatory effects of different drugs/agents with antioxidant property on endothelial expression of adhesion molecules. Cardiovasc Hematol Disord Drug Targets, 2006. 6(4): p. 279-304.
128. Bremond, A., et al., Regulation of HLA class I surface expression requires CD99 and p230/golgin-245 interaction. Blood, 2009. 113(2): p. 347-57.
129. Wilson, J. X., Mechanism of action of vitamin C in sepsis: ascorbate modulates redox signaling in endothelium. Biofactors, 2009. 35(1): p. 5-13.
130. Utoguchi, N., et al., Ascorbic acid stimulates barrier function of cultured endothelial cell monolayer. J Cell Physiol, 1995. 163(2): p. 393-9.
131. Ulfhammer, E., et al., TNF-alpha mediated suppression of tissue type plasminogen activator expression in vascular endothelial cells is NF-kappaB- and p38 MAPK- dependent. J Thromb Haemost, 2006. 4(8): p. 1781-9.
132. Xu, H., et al., Selective blockade of endothelial NF-kappaB pathway differentially affects systemic inflammation and multiple organ dysfunction and injury in septic mice. J Pathol. 220(4): p. 490-8.
133. Ding, J., et al., A pivotal role of endothelial-specific NF-kappaB signaling in the pathogenesis of septic shock and septic vascular dysfunction. J Immunol, 2009. 183(6): p. 4031-8.
134. Bowie, A. and L. A. O'Neill, Vitamin C inhibits NF kappa B activation in endothelial cells. Biochem Soc Trans, 1997. 25(1): p. 1315.
135. Carcamo, J. M., et al., Vitamin C suppresses TNF alpha-induced NF kappa B activation by inhibiting I kappa B alpha phosphorylation. Biochemistry, 2002. 41(43): p. 12995-3002.
136. Bowie, A. G. and L. A. O'Neill, Vitamin C inhibits NF-kappa B activation by TNF via the activation of p38 mitogen-activated protein kinase. J Immunol, 2000. 165(12): p. 7180-8.
137. Rodriguez-Porcel, M., et al., Chronic antioxidant supplementation attenuates nuclear factor-kappa B activation and preserves endothelial function in hypercholesterolemic pigs. Cardiovasc Res, 2002. 53(4): p. 1010-8.
138. Chade, A. R., et al., Antioxidant intervention blunts renal injury in experimental renovascular disease. J Am Soc Nephrol, 2004. 15(4): p. 958-66.
139. Levine, M., et al., Vitamin C pharmacokinetics in healthy volunteers: evidence for a recommended dietary allowance. Proc Natl Acad Sci USA, 1996. 93(8): p. 3704-9.
140. Hathcock, J. N., et al., Vitamins E and C are safe across a broad range of intakes. Am J Clin Nutr, 2005. 81(4): p. 736-45.
141. Eskurza, I., et al., Effect of acute and chronic ascorbic acid on flow-mediated dilatation with sedentary and physically active human ageing. J Physiol, 2004. 556(Pt 1): p. 315-24.

1. A method of preventing, and/or stabilizing progression of, and/or reversing Parkinson's Disease comprising induction of immunomodulatory and regenerative activity in a patient in need of therapy, wherein said method involves administration of immature dendritic cells possessing a Parkinson's Disease associated antigen together with a regenerative cell.

2. The method of claim 1, wherein said immune modulatory therapy reduces inflammation in said patient with Parkinson's Disease.

3. The method of claim 2, wherein said immune modulation is enhancement of number and/or activity of T regulatory cells.

4. The method of claim 2, wherein said inflammation is associated with a reduction of cells expressing the transcription factor FoxP3 as compared to an age matched control subject.

5. The method of claim 2, wherein said inflammation is associated with a reduction of cells expressing the cytokine interleukin-10 as compared to an age matched control subject.

6. The method of claim 2, wherein said inflammation is associated with a reduction of cells expressing the cytokine interleukin-4 as compared to an age matched control subject.

7. The method of claim 2, wherein said inflammation is associated with a reduction of cells expressing the cytokine interleukin-13 as compared to an age matched control subject.

8. The method of claim 2, wherein said inflammation is associated with a reduction of cells expressing the cytokine interleukin-20 as compared to an age matched control subject.

9. The method of claim 2, wherein said inflammation is associated with a reduction of cells expressing the cytokine interleukin-35 as compared to an age matched control subject.

10. The method of claim 2, wherein said inflammation is associated with a decrease in T regulatory cells as compared to an age matched control.

11. The method of claim 2, wherein said inflammation is associated with a decrease in myeloid suppressor cells as compared to an age matched control.

12. The method of claim 2, wherein said inflammation is associated with a decrease in TIM-1 expressing B cells as compared to an age matched control.

13. The method of claim 2, wherein said inflammation is associated with a decrease in interleukin-10 expressing B cells as compared to an age matched control.

14. The method of claim 2, wherein said inflammation is associated with a decrease in B regulatory cells as compared to an age matched control.

15. The method of claim 2, wherein said inflammation is associated with an increase in cells expressing interferon gamma as compared to an age matched control.

16. The method of claim 2, wherein said inflammation is associated with an increase in cells expressing TNF-alpha as compared to an age matched control.

17. The method of claim 2, wherein said inflammation is associated with an increase in cells expressing interleukin-1 as compared to an age matched control.

18. The method of claim 2, wherein said inflammation is associated with an increase in cells expressing interleukin-2 as compared to an age matched control.

19. The method of claim 2, wherein said inflammation is associated with an increase in cells expressing interleukin-6 as compared to an age matched control.

20. The method of claim 2, wherein said inflammation is associated with an increase in cells expressing interleukin-18 as compared to an age matched control.
